(19) United States
(12) Patent Application Publication
(10) Pub. No.: US 2025/0185933 A1
(43) Pub. Date: Jun. 12, 2025

(54) HEART RATE DETECTION APPARATUS AND HEAD-MOUNTED DEVICE

(71) Applicant: Beijing Zitiao Network Technology Co., Ltd., Beijing (CN)

(72) Inventors: Zhao FENG, Beijing (CN); Zishang WANG, Beijing (CN)

(21) Appl. No.: 18/927,114

(22) Filed: Oct. 25, 2024

(30) Foreign Application Priority Data
Dec. 6, 2023 (CN) 202311667372.5

(51) Int. Cl. A61B 5/024; A61B 5/00

(52) U.S. Cl. CPC A61B 5/02438; A61B 5/6803; A61B 5/742

(57) ABSTRACT

The present application discloses a heart rate detection apparatus and a head-mounted device, wherein the heart rate detection apparatus comprises: a face contact structure configured to abut against a user's face, the face contact structure being provided with a first mounting hole located in an area of the face contact structure opposite to a user's forehead; a mounting bracket disposed in the first mounting hole of the face contact structure; and a heart rate sensor mounted on the mounting bracket and having a detection end disposed facing the user's face, the heart rate sensor being further electrically connected to a processor of the head-mounted device.

### CROSS REFERENCE TO RELATED APPLICATIONS

This present application claims the benefit of priority to Chinese Application No. 202311667372.5, filed on Dec. 6, 2023, the entire contents of which are incorporated herein by reference.

### TECHNICAL FIELD

The present application pertains to the technical field of electronic devices, and in particular to a heart rate detection apparatus and a head-mounted device.

### BACKGROUND

With the continuous development of science and technology, technologies such as virtual reality (VR) have gradually entered various industries. Taking VR devices as an example, users can use VR devices, such as head-mounted devices, to watch virtual reality scenes.

### SUMMARY

Embodiments of the present application provide a heart rate detection apparatus and a head-mounted device.

In a first aspect, the present application provides a heart rate detection apparatus, for use with a head-mounted device, comprising: a face contact structure configured to abut against a user's face, the face contact structure comprising a first mounting hole located in an area of the face contact structure opposite to a user's forehead; a mounting bracket disposed in the first mounting hole of the face contact structure; a heart rate sensor mounted on the mounting bracket and having a detection end disposed facing the user's face, the heart rate sensor being further electrically connected to a processor of the head-mounted device.

In some embodiments, the mounting bracket is slidably disposed in the first mounting hole; the heart rate detection apparatus further comprises an elastic member having one end abutting against the mounting bracket, the elastic member being configured to push the mounting bracket and the heart rate sensor in a direction facing the user's face.

In some embodiments, in the first mounting hole is fixedly disposed a shaft seat and is further disposed a mounting shaft, one end of the mounting shaft being slidably disposed in a central hole of the shaft seat, and the other end of the mounting shaft being fixedly connected to the mounting bracket; one end of the elastic member facing away from the mounting bracket abuts against the shaft seat.

In some embodiments, the shaft seat comprises a mounting sleeve and a limit ring, wherein the limit ring is connected to one end of the mounting sleeve facing the elastic member; an aperture of a part of the central hole of the shaft seat located in the mounting sleeve is greater than that of a part of the central hole of the shaft seat located in the limit ring; the mounting shaft is provided with a flange extending in its radial direction at one end facing the shaft seat, the flange being located in the mounting sleeve and having an outer diameter greater than the aperture of the part of the central hole of the shaft seat located in the limit ring.

In some embodiments, on one side of the face contact structure facing away from the user's face is further provided a quick release part, which is configured to detachably connect the face contact structure to a host of the head-mounted device;
wherein the quick release part comprises at least one of the following: a magnetic attraction member or a snap member.

In some embodiments, the heart rate detection apparatus further comprises a flexible circuit board, which is located at least in part in the first mounting hole and is electrically connected to the heart rate sensor, the flexible circuit board being further electrically connected to the processor of the head-mounted device through a pogo pin.

In some embodiments, the mounting bracket comprises: a mounting body having a first end and a second end connected to each other, the first end being disposed facing the user's face; the first end being provided with a second mounting hole in which the heart rate sensor is located, the second end being provided with a shaft hole that is in sliding fit with one end of the mounting shaft facing the user's face.

In some embodiments, the mounting body further comprises: a partition plate disposed at a joint of the first end and the second end, which is configured to isolate the second mounting hole from the shaft hole.

In some embodiments, the mounting bracket is connected with a light-transmitting lens, which shields on one side of the heart rate sensor facing the user's face and is provided to at least cover an orifice of the second mounting hole.

In some embodiments, an outer periphery of the mounting body is further connected with a first extension extending outward, one side of which facing away from the mounting body is further connected with a second extension, the second extension being provided to fold in the direction facing the user's face relative to the first extension, so that the first extension, the second extension and a part of the mounting body enclose a mounting slot; the light-transmitting lens comprises: a lens body configured to cover an orifice of the second mounting hole at one end facing the user's face, the lens body being provided with a flap at an edge, which is folded in a direction facing away from the user's face, the flap being inserted into the mounting slot.

In some embodiments, the first mounting hole is disposed in an upper portion of the face contact structure, and is located in a middle area of the face contact structure.

In some embodiments, the heart rate detection apparatus further comprises a protective cover, which detachably covers one end of the first mounting hole facing away from the user's face.

In a second aspect, the present application provides a head-mounted device, which comprises a host and a heart rate detection apparatus as described in any one of the foregoing, the heart rate detection apparatus being connected to the host.

For the heart rate detection apparatus and the head-mounted device provided by the present application, a face contact structure is provided, which is configured to abut against a user's face, a first mounting hole being disposed in the face contact structure and located in an area of the face contact structure opposite to a user's forehead; a mounting bracket is provided, which is disposed in the first mounting hole of the face contact structure; a heart rate sensor is provided, which is mounted on the mounting bracket and has a detection end disposed facing the user's face, the heart rate sensor being further electrically connected to a processor of the head-mounted device.

### BRIEF DESCRIPTION OF THE DRAWINGS

In order to more clearly illustrate the technical solutions in the embodiments of the present application or the related art, the drawings to be used in the description of the embodiments or the related art will be briefly introduced below. Apparently, the drawings in the following description are some embodiments of the present application, and for those skilled in the art, other drawings can be obtained based on these drawings without inventive effort. In the drawings:

FIG. 1 is a schematic view of an application scenario of a heart rate detection apparatus provided by an embodiment of the present application;

FIG. 2 is a first schematic structural view of a heart rate detection apparatus provided by an embodiment of the present application;

FIG. 3 is a schematic, partial cross-sectional view of a heart rate detection apparatus (in a state of being tightly attached to a user's forehead) provided by an embodiment of the present application in a direction A-A in FIG. 2;

FIG. 4 is a schematic, partial cross-sectional view of a heart rate detection apparatus provided by another embodiment of the present application in the direction A-A in FIG. 2;

FIG. 5 is an enlarged schematic view of part B in FIG. 4;

FIG. 6 is a schematic view of a state in which a heart rate detection apparatus provided by an embodiment of the present application is tightly attached to the user's forehead;

FIG. 7 is a schematic exploded view of a heart rate detection apparatus provided by an embodiment of the present application;

FIG. 8 is a second schematic structural view of a heart rate detection apparatus provided by an embodiment of the present application.

### ILLUSTRATION OF REFERENCE NUMERALS

1—heart rate detection apparatus; 11—face contact structure; 11a—first mounting hole; 111—foam body; 112—foam support; 113—mounting shaft; 1131—flange; 114—shaft seat; 1141—mounting sleeve; 1142—limit ring; 115—magnetic attraction member; 12—mounting bracket; 121—mounting body; 1211—first end; 1212—second end; 1213—partition plate; 122—first extension; 123—second extension; 122a—mounting slot; 13—heart rate sensor; 14—elastic member; 15—flexible circuit board; 151—electrical contact; 31—pogo pin; 16—light-transmitting lens; 161—lens body; 162—flap; 17—protective cover; 18—screw; 3—host.

### DETAILED DESCRIPTION

The embodiments of the present application will be described below in detail, examples of which are shown in the accompanying drawings. The embodiments described below with reference to the drawings are exemplary and are intended to be used for explaining the present application, but not to be construed as limiting the present application.

The names of messages or information exchanged between multiple devices in the embodiments of the present disclosure are for illustrative purposes only and are not used to limit the scope of such messages or information.

The embodiments of the present application can be applied to extended Reality (XR), such as Virtual Reality (VR), Augmented Reality (AR), Mixed Reality (MR) and other various application scenarios.

Taking virtual reality (VR) as an example, VR is a technology for creating and experiencing a virtual world, which implements a simulation of a fused, interactive three-dimensional dynamic view and physical behaviors of a virtual environment based on multi-source information (e.g., visual perception, auditory perception, tactile perception, motion perception, and even taste perception, olfactory perception, etc.), allowing users to immerse themselves in the simulated virtual reality environment, thereby implementing application in a plurality of virtual environments such as maps, games, videos, education, healthcare, simulations, collaborative training, sales, assisted manufacturing, maintenance, and repair.

A terminal that achieves virtual reality effects is generally called a VR device, which may be typically provided in a form of a head-mounted device. The head-mounted device generally comprises: a host, a face-sticking foam and a fixing member, wherein the face-sticking foam is connected to one side of the host to contact a user's face, and the fixing member is configured to fix the host to the user's face, so that two display modules in the host are opposite to the user's eyes respectively, thus enabling the user to watch the virtual reality scene. However, the head-mounted devices in the related art have a relatively single overall function.

In order to overcome the above problem, an embodiment of the present application provides a heart rate detection apparatus for a head-mounted device; by arranging a first mounting hole in an area of a face contact structure corresponding to a user's forehead and mounting a heart rate sensor in the first mounting hole through a mounting bracket, the heart rate sensor close to the user's forehead can detect the user's heart rate, thus enriching the functions of the head-mounted device provided with the heart rate detection apparatus.

The technical solution of the present application and how the technical solution of the present application solves the above technical problem will be described in detail with specific embodiments. The following specific embodiments can be combined with each other, and the same or similar concepts or processes may not be repeated in some embodiments. The embodiments of the present application will be described below with reference to the accompanying drawings.

For convenience of description, in this embodiment, when the head-mounted device is in a state in which it is worn on the user's head, the direction of the head-mounted device facing the user is front, the direction facing the ground is down, and the up-down direction is vertical.

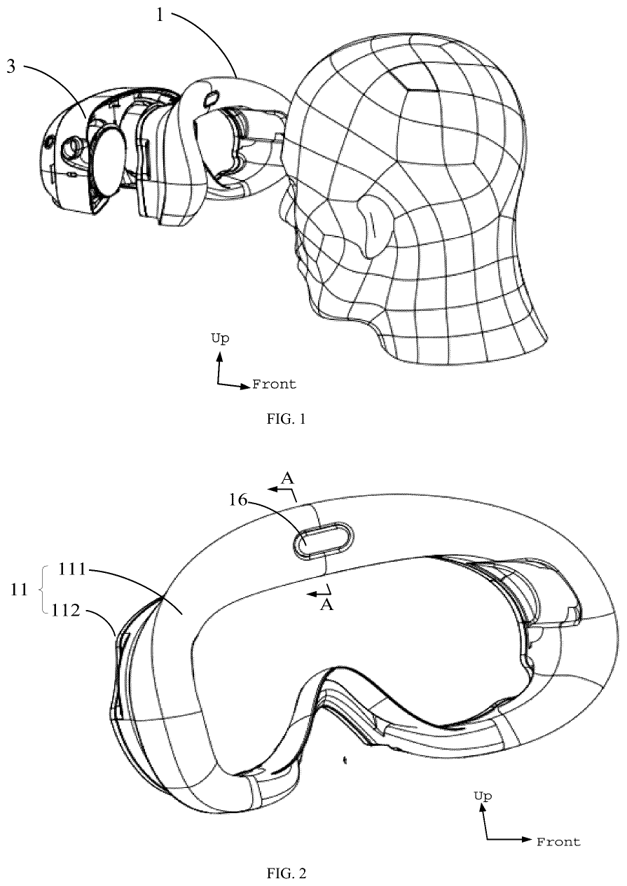

Referring to FIGS. 1-3, an embodiment of the present application provides a heart rate detection apparatus 1 for a head-mounted device, comprising: a face contact structure 11, a front side of which is configured to abut against a user's face, wherein in the face contact structure 11 is disposed a first mounting hole 11a, which is located in an area of the face contact structure 11 opposite to a user's forehead; a mounting bracket 12 disposed in the first mounting hole 11a of the face contact structure; a heart rate sensor 13 mounted on the mounting bracket 12 and having a detection end disposed facing the front side of the face contact structure 11, the heart rate sensor 13 being further electrically connected to a processor of the head-mounted device.

The face contact structure 11 is configured to connect to a front side of a host 3 of the head-mounted device, so as to directly contact the user's face when the user wears the head-mounted device, thus improving the wearing comfort of the user.

Illustratively, the face contact structure 11 can be a face-sticking foam, which may specifically include a foam body 111 and a foam support 112, wherein the foam body 111 is fixed to the foam support 112, and the foam support 112 is connected to the host 3 of the head-mounted device. The foam body 111 may be fixedly connected to the foam support 112 through a bonding or snap-fit connection. The foam support 112 may be connected to the host 3 of the head-mounted device through a fastening or snap-fit connection. In other embodiments, the face contact structure 11 may also be a structure made of other materials such as silica gel or the like.

In some embodiments, the face contact structure 11 is provided with a first mounting hole 11a in which the heart rate sensor 13 is mounted. The first mounting hole 11a may be disposed in an upper and relatively centered area of the face contact structure 11. For example, a vertical central line of the first mounting hole 11a may coincide with a vertical central line of the face contact structure 11, or have a preset spacing therefrom. In this way, the heart rate sensor 13 can be tightly attached to the user's forehead with abundant capillaries, thereby improving the accuracy of heart rate detection.

Illustratively, the first mounting hole 11a is through disposed in a front-rear direction. In some examples, the first mounting hole 11a may be disposed on the foam body 111. In other examples, the foam body 111 is provided with a through-hole, and the face contact structure bracket 112 is provided with a mounting seat, the first mounting hole 11a being disposed on the mounting seat.

In other embodiments, the first mounting hole 11a may also be mounted in an upper and relatively left or relatively right area of the face contact structure 11.

The heart rate sensor 13 may be mounted in the first mounting hole 11a through the mounting bracket 12. The heart rate sensor 13 can be fixed to a first end of the mounting bracket 12. In some examples, the heart rate sensor 13 is fixed to the first end of the mounting bracket 12 through an insertion, snap-fit or bonding connection.

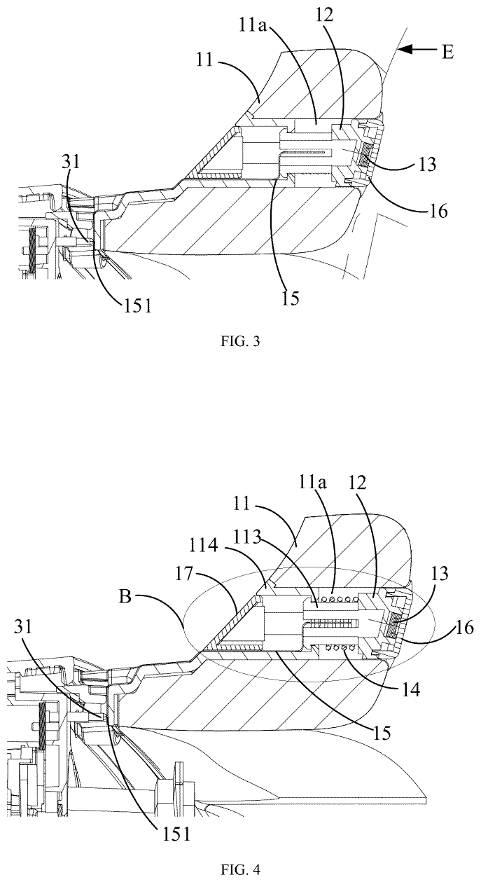

In some embodiments, as shown in FIG. 3, the mounting bracket 12 can be mounted into the first mounting hole 11a through an interference fit or a bonding or a snap-fit connection. The heart rate sensor 13 can be tightly attached to the user's forehead E by means of the elasticity of the foam body itself in the face contact structure 11.

Referring to FIGS. 4-6, in other embodiments, the mounting bracket 12 is slidably disposed in the first mounting hole 11a in which an elastic member 14 is disposed; the elastic member 14 may expand and contract in an axial direction of the first mounting hole 11a, and one end of the elastic member 14 may abut against the mounting bracket 12, and the elastic member 14 may push the mounting bracket 12 and the heart rate sensor 13 forward, so that the heart rate sensor 13 can be tightly attached to the user's forehead, thereby being able to adapt to the use requirements of users with different head types. The elastic member 14 may be a spring or an elastic rubber sleeve.

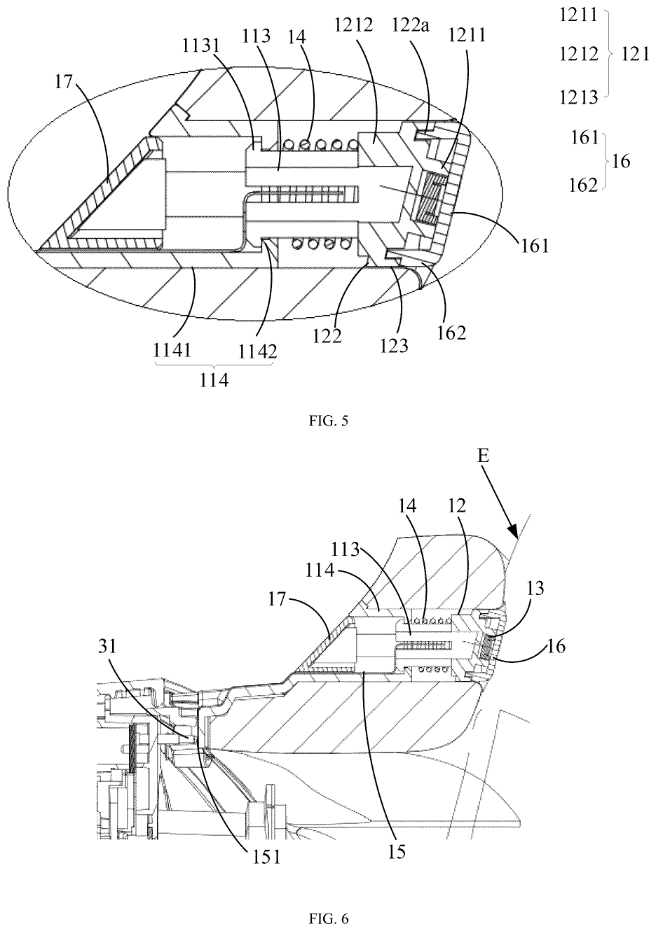

When the heart rate detection apparatus 1 is in a state in which it is not attached to the user's forehead, the elastic member 14 may be in a compressed state or an original state. As shown in FIG. 6, when the heart rate detection apparatus 1 is in the state in which it is attached to the user's forehead E, the user's forehead E pushes back against the mounting bracket 12 and the heart rate sensor 13, and compresses the elastic member 14, so that the heart rate sensor 13 is tightly attached to the user's forehead E under the elastic potential energy of the elastic member 14.

In some examples, a shaft seat 114 is fixedly disposed in the first mounting hole 11a, and a mounting shaft 113 is further disposed in the first mounting hole 11a and is slidably inserted into a central hole of the shaft seat 114. The axial direction of the shaft seat 114 and the mounting shaft 113 can be parallel to the axial direction of the first mounting hole 11a. The shaft seat 114 may be integrally provided with the foam support 112, or the shaft seat 114 is an independent component and fixed into the first mounting hole 11a.

A front end of the mounting shaft 113 is fixedly connected to the mounting bracket 12. The mounting shaft 113 is externally sleeved with the elastic member 14, both ends of which are respectively abutted against the shaft seat 114 and the mounting bracket 12, and the elastic member 14 can push the mounting bracket 12 and the heart rate sensor 13 forward, so that the heart rate sensor 13 can be tightly attached to the user's forehead, thereby being able to adapt to the use requirements of users with different head types.

Illustratively, the shaft seat 114 may have a mounting sleeve 1141 and a limit ring 1142 disposed at a front end of the mounting sleeve 1141, and the limit ring 1142 can be coaxially disposed with the mounting sleeve 1141, and an aperture of a part of a central hole of the shaft seat 114 located in the mounting sleeve 1141 is greater than that of a part of the central hole located in the limit ring 1142. A rear end of the mounting shaft 113 is provided with a flange 1131 extending in its radial direction, the flange 1131 being located in the mounting sleeve 1141 and having an outer diameter greater than the aperture of the part of the central hole located in the limit ring 1142. One end of the elastic member 14 may abut against the second end 1212 of the mounting bracket 12, and the other end of the elastic member may abut against the limit ring 1142 of the shaft seat. With the above arrangement, by abutting against the flange 1131 of the mounting shaft 113, the limit ring 1142 can define a limit position where the mounting bracket 12 and the heart rate sensor 13 slide forward, so as to prevent the mounting bracket 12 and the heart rate sensor 13 from detaching from an orifice of the first mounting hole 11a at the front end.

The rear end of the mounting sleeve 1141 may be provided with a first extending edge which extends in a direction away from the central axis of the mounting sleeve 1141, and a shallow slot matched with the extending edge is provided on the rear side of the foam body 111 of the face contact structure 11. The specific shape of the first extending edge can be set according to actual needs.

Illustratively, the mounting shaft 113 may be in a stepped shape, with a first shaft section and a second shaft section connected to each other, the first shaft section being connected to a front end of the second shaft section and having a shaft diameter less than that of the second shaft section. The mounting bracket 12 is fixedly connected to the first shaft section. The elastic member 14 is sleeved outside the second shaft section. The rear end of the mounting shaft 113, i.e., the rear end of the second shaft section, is provided with a flange 1131. The first shaft section, the second shaft section and the flange 1131 of the mounting shaft 113 can be integrally provided; alternatively, the mounting shaft comprises a shaft body and a shaft sleeve sleeved at a rear end of the shaft body, a part of the shaft body not sleeved with the shaft sleeve constitutes the first shaft section, a part of the shaft body sleeved with the shaft sleeve constitutes the second shaft section, and a rear end of the shaft body is provided with a flange 1131.

With the heart rate detection apparatus 1 provided by this embodiment, when the heart rate detection apparatus 1 is in the state in which it is not attached to the user's forehead, the elastic member 14 can be in a compressed state, and the flange 1131 of the mounting shaft 113 abuts against the limit ring 1142 of the shaft seat 114; as shown in FIG. 6, when the heart rate detection apparatus 1 is in a state in which it is attached to the user's forehead E, the mounting bracket 12 and the heart rate sensor 13 can slide backwards in the first mounting hole 11a according to a contour of the user's head, such as the user's forehead, and further compress the elastic member 14, that is, the user's forehead will push back against the mounting bracket 12 and the heart rate sensor 13 and further compress the elastic member 14, so that the detection end of the heart rate sensor 13 can be tightly attached to the user's forehead under the elastic effect of the elastic member 14, thereby being able to adapt to the use requirements of users with different head types.

In other embodiments, the mounting shaft 113 can be fixedly connected to the shaft seat 114, and the mounting bracket 12 can be slidably disposed at the front end of the mounting shaft 113, so that the mounting bracket 12 and the heart rate sensor 13 slide in the axial direction of the mounting shaft 113. The elastic member 14 can be sleeved outside the mounting shaft 113 and abutted between the mounting bracket 12 and the shaft seat 114.

The heart rate sensor 13 is substantially in a columnar shape. In some examples, the heart rate sensor 13 can be electrically connected to a processor of the head-mounted device, so that a battery module of the head-mounted device can supply power to the heart rate sensor 13 and enable the heart rate sensor 13 to perform data communication with the processor. In other examples, the heart rate sensor 13 can perform data communication with the processor through wireless communication, and the battery module for supplying power to the heart rate sensor 13 is disposed in the face contact structure 11.

After the heart rate information detected by the heart rate sensor 13 is transmitted to the processor, the processor can prompt the user according to the heart rate information, such as displaying it, so that the user can know his own heart rate state in time. For example, for fitness enthusiasts, the exercise intensity can be adjusted in a targeted manner through a heart rate prompt. In other examples, the processor can also transmit the heart rate information to other terminals or wearable devices, such as sports watches or mobile phones, which are communicatively connected to the head-mounted device, so as to facilitate user viewing.

For the heart rate detection apparatus 1 provided by this embodiment, which is used for a head-mounted device, a face contact structure 11 is provided, which is configured to abut against a user's face facing the user's front side, a first mounting hole 11a being disposed in the face contact structure 11 and located in an area of the face contact structure 11 opposite to a user's forehead; a mounting bracket 12 is provided, which is disposed in the first mounting hole 11a of the face contact structure; a heart rate sensor 13 is provided, which is mounted on the mounting bracket 12 and has a detection end disposed facing the front side of the face contact structure 11, the heart rate sensor 13 being further electrically connected to a processor of the head-mounted device. In this way, when the heart rate detection apparatus 1 is attached to the user's face, the heart rate sensor 13 close to the user's forehead can detect the user's heart rate, thus enriching the functions of the head-mounted device provided with the heart rate detection apparatus.

Referring to FIGS. 4-6, in some embodiments, the mounting bracket 12 comprises a mounting body 121 with a first end 1211 and a second end 1212 connected to each other, the first end 1211 being provided with a second mounting hole in which the heart rate sensor 13 is located, the second end 1212 being provided with a shaft hole that is matched with the front end of the mounting shaft 113.

Illustratively, an outer diameter of the first end 1211 may be less than that of the second end 1212, which is beneficial to the compactness of the heart rate detection apparatus 1. The axial direction of the first end 1211 may have a preset included angle with the axial direction of the second end 1212, for example, the first end 1211 of the mounting body 121 may be coaxially disposed with the mounting shaft 113; the axial direction of the second end 1212 of the mounting body 121 is inclined downward relative to the axial direction of the first end 1211.

In some examples, the mounting body 121 further comprises a partition plate 1213, which is disposed at a joint of the first end 1211 and the second end 1212 to isolate the second mounting hole from the shaft hole. The partition plate 1213 may be an inclined plate, which can be perpendicular to the axial direction of the second end 1212 of the mounting body 121.

The mounting bracket 12 is further connected with a light-transmitting lens 16, which shields on the front side of the heart rate sensor 13 and at least covers an orifice of the second mounting hole. In this way, the light-transmitting lens 16, the partition plate 1213 and the first end 1211 can enclose a relatively closed receiving space in which the heart rate sensor 13 is disposed, thus being able to play a role in sealing protection.

The light-transmitting lens 16 may be made of a light-transmitting material for transmitting the light emitted from the heart rate sensor 13. The light-transmitting lens 16 may be fixedly connected to the mounting bracket 12 so that the light-transmitting lens 16 can slide with the mounting bracket 12 in the axial direction of the first mounting hole 11a.

When the heart rate detection apparatus 1 is in the state in which it is not attached to the user's forehead, the elastic member 14 may be in a compressed state, and the flange 1131 of the mounting shaft 113 abuts against the limit ring 1142 of the shaft seat 114; as shown in FIG. 6, when the heart rate detection apparatus 1 is in the state in which it is attached to the user's forehead E, the user's forehead pushes back against the mounting bracket 12 and the heart rate sensor 13 through the light-transmitting lens 16, and further compresses the elastic member 14, so that the light-transmitting lens 16 can be tightly attached to the user's forehead E under the elastic potential energy of the elastic member 14.

In some examples, an outer periphery of the mounting body 121 is further connected with a first extension 122 extending outwards, and one side of the first extension 122 facing away from the mounting body 121 is further connected with a second extension 123, which is folded forward relative to the first extension 122, so that the first extension 122, the second extension 123 and part of the mounting body 121 enclose a mounting slot 122a.

The first extension 122 may be connected to a peripheral surface of a middle portion of the mounting body 121, for example, the first extension 122 may be connected to an area of the second end 1212 close to the first end 1211, and the first extension 122 may be disposed extending in a radial direction of the mounting body 121. The first extension 122 may be disposed parallel to the radial direction of the mounting body 121, or the first extension 122 may have a preset included angle with the radial direction of the mounting body 121. The first extension 122 may be disposed extending in a circumferential direction of the mounting body 121, or may be a protruding structure distributed in the circumferential direction of the mounting body 121.

One side of the first extension 122 facing away from the mounting body 121 is connected with the second extension 123, which may be disposed parallel to the axial direction of the first mounting hole 11a. The second extension 123 may be disposed extending in the circumferential direction of the mounting body 121, or may be a protruding structure distributed in the circumferential direction of the mounting body 121.

The first extension 122, the second extension 123 and part of the mounting body 121 enclose a mounting slot 122a, which is matched with the light-transmitting lens 16. The light-transmitting lens 16 comprises a lens body 161, which is configured to cover an orifice of the second mounting hole at the front end. In this way, the lens body 161, the partition plate 1213 and the first end 1211 can enclose a relatively closed receiving space in which the heart rate sensor 13 is disposed, thus being able to play a role in sealing protection.

Illustratively, the size of the lens body 161 may be slightly smaller than an orifice of the first mounting hole 11a at the front end, so that the light-transmitting lens 16 can smoothly slide with the mounting bracket 12 in the axial direction of the first mounting hole 11a, and can also provide a shielding protection function, thus reducing dirt such as dust deposited at the orifice of the first mounting hole 11a. The shape of the lens body 161 can be adapted to the shape of the orifice of the first mounting hole 11a at the front end; for example, the orifice of the first mounting hole 11a at the front end is oblong, and the lens body 161 is also oblong.

The lens body 161 is provided with a flap 162 at an edge, which is folded backward, the flap 162 being inserted into the mounting slot 122a. The flap 162 can be fixedly disposed in the mounting slot 122a through an interference fit, or a bonding or snap-fit connection.

In some examples, the flap 162 faces a surface of a hole wall of the first mounting hole 11a, and is recessed in a direction away from the hole wall of the first mounting hole 11a, in an area away from the lens body 161, so as to form a mating surface that interfaces with the second extension 123 of the mounting bracket 12. In this way, a connection surface of the light-transmitting lens 16 and the mounting bracket 12 is complex, making a sealing path between the light-transmitting lens 16 and the mounting bracket 12 complex, so that the sealing protection effect on the heart rate sensor 13 can be improved.

In this example, the light-transmitting lens 16 is provided, which can not only provide sealing protection for the heart rate sensor 13, but also provide shielding protection for the first mounting hole 11a.

In other examples, the lens body 161 may also be directly bonded to a front end surface of the first end 1211 of the mounting body 121, or the first end 1211 of the mounting body 121 is provided with a socket, and a rear surface of the lens body 161 facing the first end 1211 is provided with an insertion post, which can be interference fit with the socket.

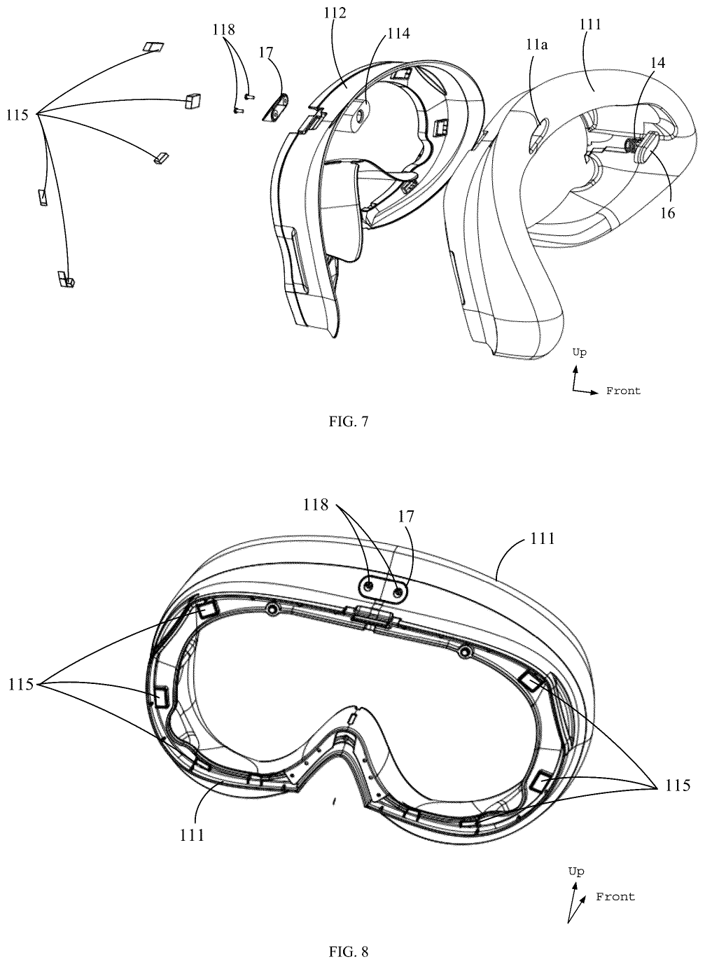

Referring to FIG. 7 and FIG. 8, in some embodiments, the heart rate detection apparatus 1 further comprises a protective cover 17 that detachably covers the rear end of the first mounting hole 11a. The protective cover 17 can protect components located in the first mounting hole 11a.

Illustratively, the protective cover 17 may include a cover body shielding at an orifice of the first mounting hole 11a at the rear end. The specific shape of the cover body is adapted to the orifice of the first mounting hole 11a at the rear end; for example, when the orifice at the rear end of the first mounting hole 11a is inclined, the cover body is also inclined; when the orifice at the rear end of the first mounting hole 11a is an oblong hole, the cover body may also be in the shape of an oblong plate.

The cover body is connected with a second extending edge, which may extend along the circumferential direction of the protective cover 17 and is inserted into the first mounting hole 11a. When the shaft seat 114 is provided at the first mounting hole 11a, the second extending edge is inserted into the central hole of the shaft seat 114.

The front surface of the cover body may also be connected with a mounting post extending forward, the mounting post passes through a hole that is disposed through the cover body, and within the through-hole is provided a screw 18 therethrough. The aperture of a part of the through-hole located in the cover body may be greater than that of a part of the through-hole located in the mounting post, so that the head of the screw 18 can be received in the part of the through-hole located in the cover body.

The first mounting hole 11a is an oblong hole, and the shape of a part of the shaft seat 114 inserted into the first mounting hole 11a is adapted to the shape of the first mounting hole 11a. For example, the cross-section of the shaft seat 114 perpendicular to its axial direction may be an oblong surface. The central area of the shaft seat 114 is provided with a central hole through which the mounting shaft 113 is disposed. The rear end of the shaft seat 114 may also be provided with a threaded hole, which is disposed apart from the central hole. The screw 18 disposed through the through-hole can be matched with the threaded hole of the shaft seat 114 at the rear end.

Referring to FIG. 7 and FIG. 8, in some embodiments, the rear end of the face contact structure 11 is further provided with a quick release part, which is configured to detachably connect the face contact structure 11 to the host 3 of the head-mounted device, thus facilitating rapid disassembly and assembly by the user, so that the heart rate detection apparatus 1 can be used as an optional component for the user to choose. The quick release part includes at least one of the following: a magnetic attraction member 115 and a snap member (not shown).

In some examples, the quick release part includes a plurality of magnetic attraction members 115 distributed at intervals, and the host 3 of the head-mounted device is provided with a magnetic attraction area matched with the magnetic attraction member 115; the face contact structure 11 can be detachably connected to the host 3 of the head-mounted device through a magnetic attraction force of the magnetic attraction member 115 and the magnetic attraction area, so that the heart rate detection apparatus 1 can be used as an optional component for the user to choose.

The magnetic attraction member 115 can be a block-shaped magnet, and the part of the host 3 of the head-mounted device located in the magnetic attraction area and in contact with the magnetic attraction member 115 can be made of a metal material that can be attracted by the magnet.

The number and distribution of the magnetic attraction members 115 can be set according to actual needs. Illustratively, the plurality of magnetic attraction members 115 may be symmetrically distributed about a central plane of the face contact structure 11 in an up-down direction. For example, there may be three magnetic attraction members 115, one of which may be disposed in a middle area of an upper portion of the face contact structure 11, and the other two may be disposed on a lower portion of the face contact structure 11 and close to the left and right sides respectively. There may be at least four magnetic attraction members 115, which may be respectively disposed close to corners of the face contact structure 11.

In other examples, the quick release part includes a plurality of snap members distributed at intervals, and the host 3 of the head-mounted device is provided with a snap hole matched with the snap member. Alternatively, the quick release part includes a magnetic attraction member and a snap member, and the specific number of magnetic attraction members and snap members is set according to actual needs.

In some examples, the heart rate detection apparatus 1 further comprises a flexible circuit board 15 that is located at least in part in the first mounting hole 11a and electrically connected to the heart rate sensor 13, the flexible circuit board 15 being further electrically connected to the processor of the head-mounted device through a pogo pin 31.

The mounting shaft 113 may be provided with an avoidance hole in communication with the partition plate 1213 of the mounting bracket 12, and part of the flexible circuit board 15 may be located in the avoidance hole, so that the flexible circuit board 15 is electrically connected to the heart rate sensor 13. The avoidance hole of the mounting shaft 113 may further be in communication with the central hole of the shaft seat 114, so that the flexible circuit board 15 can pass through the central hole and be electrically connected to the processor of the head-mounted device. The extension lines of the flexible circuit board 15 can be set according to actual needs, which is not specifically limited here in this embodiment. The extension lines of the flexible circuit board 15 need to make the flexible circuit board 15 (except electrical contact 151) not exposed in the heart rate detection apparatus 1 as much as possible.

The flexible circuit board 15 is provided with an electrical contact 151, and the processor of the head-mounted device is provided with a pogo pin 31. When the heart rate detection apparatus 1 is connected to the host 3, the flexible circuit board 15 is electrically connected to the processor of the head-mounted device through the pogo pin 31.

In this embodiment, with the above arrangement, the heart rate detection apparatus 1 can be used as an optional component for the user to choose, and the electrical connection reliability of the heart rate sensor 13 can be ensured; during use by the user, the user may choose the face contact structure 11 without the heart rate detection function to assemble with the host 3 of the head-mounted device, or choose the heart rate detection apparatus 1 in the above embodiment to assemble with the host 3 of the head-mounted device.

The embodiments of the present application further provide a head-mounted device, which comprises a host 3 and a heart rate detection apparatus 1 in any of the aforementioned embodiments, the heart rate detection apparatus 1 being connected to the host 3. The structure, function and implementation process of the heart rate detection apparatus 1 are the same as those of the previous embodiments, and the details are not repeated here in this embodiment. In addition, for the head-mounted device, the parts not explained in this embodiment can be set according to actual needs.

It should be understood that the orientation or positional relations indicated by the terms “up”, “down”, “front”, “rear”, “left”, “right”, “vertical”, “horizontal”, “top”, “bottom”, “in”, “out”, “clockwise”, “counterclockwise”, “axial”, “radial”, “circumferential” and the like are based on the orientation or positional relations shown in the drawings, are merely for convenience of describing the present application and simplifying the description, and are not intended to indicate or imply that the device or elements referred to must have a specific orientation, be constructed and operated in a specific orientation, and therefore cannot be construed as limiting the present application. In the description of the present application, “a plurality of” means two or more.

As used herein, the term “comprising” and variations thereof are open-ended, i.e., “comprising but not limited to”. The term “based on” is “based at least in part on”. The term “one embodiment” means “at least one embodiment”; the term “another embodiment” means “at least one further embodiment”; the term “some embodiments” means “at least some embodiments”. The relevant definitions of other terms will be given below.

It should be noted that the concepts such as “first” and “second” mentioned in the present application are merely used to distinguish different apparatuses, modules, or units, and are not intended to limit the order of functions performed by the apparatuses, modules, or units or their mutual dependencies.

It should be noted that, the modification by “a” or “a plurality of” referred to in the present application is meant to be illustrative and non-limiting, and should be construed as “one or more,” unless otherwise expressly indicated in the context, as will be appreciated by those skilled in the art.

The above description is only for specific embodiments of the present application, but the scope of protection of the present application is not limited thereto, and any person skilled in the art may easily conceive of variations or substitutions within the technical scope revealed by the present application, which should be covered within the scope of protection of the present application. Therefore, the scope of protection of the present application should be based on the protection scope of the claims.

What is claimed is:

1. A heart rate detection apparatus, for use with a head-mounted device, comprising:
a face contact structure configured to abut against a user's face, the face contact structure comprising a first mounting hole located in an area of the face contact structure opposite to a user's forehead;
a mounting bracket disposed in the first mounting hole of the face contact structure; and
a heart rate sensor mounted on the mounting bracket and having a detection end disposed facing the user's face, the heart rate sensor being further electrically connected to a processor of the head-mounted device.

2. The heart rate detection apparatus according to claim 1, wherein the mounting bracket is slidably disposed in the first mounting hole;
the heart rate detection apparatus further comprises an elastic member having one end abutting against the mounting bracket, the elastic member being configured to push the mounting bracket and the heart rate sensor in a direction facing the user's face.

3. The heart rate detection apparatus according to claim 2, wherein in the first mounting hole is fixedly disposed a shaft seat and is further disposed a mounting shaft, one end of the mounting shaft being slidably disposed in a central hole of the shaft seat, and the other end of the mounting shaft being fixedly connected to the mounting bracket; one end of the elastic member facing away from the mounting bracket abuts against the shaft seat.

4. The heart rate detection apparatus according to claim 3, wherein the shaft seat comprises a mounting sleeve and a limit ring, wherein the limit ring is connected to one end of the mounting sleeve facing the elastic member; an aperture of a part of the central hole of the shaft seat located in the mounting sleeve is greater than that of a part of the central hole of the shaft seat located in the limit ring;
the mounting shaft is provided with a flange extending in its radial direction at one end facing the shaft seat, the flange being located in the mounting sleeve and having an outer diameter greater than the aperture of the part of the central hole of the shaft seat located in the limit ring.

5. The heart rate detection apparatus according to claim 1, wherein on one side of the face contact structure facing away from the user's face is further provided a quick release part, which is configured to detachably connect the face contact structure to a host of the head-mounted device;
wherein the quick release part comprises at least one of the following: a magnetic attraction member or a snap member.

6. The heart rate detection apparatus according to claim 5, further comprising a flexible circuit board, which is located at least in part in the first mounting hole and is electrically connected to the heart rate sensor, the flexible circuit board being further electrically connected to the processor of the head-mounted device through a pogo pin.

7. The heart rate detection apparatus according to claim 1, wherein the mounting bracket comprises a mounting body having a first end and a second end connected to each other, the first end being disposed facing the user's face; the first end being provided with a second mounting hole in which the heart rate sensor is located, the second end being provided with a shaft hole that is in sliding fit with one end of the mounting shaft facing the user's face.

8. The heart rate detection apparatus according to claim 7, wherein the mounting bracket is connected with a light-transmitting lens, which shields on one side of the heart rate sensor facing the user's face and at least covers an orifice of the second mounting hole.

9. The heart rate detection apparatus according to claim 8, wherein an outer periphery of the mounting body is further connected with a first extension extending outward, one side of which facing away from the mounting body is further connected with a second extension, the second extension being folded in the direction facing the user's face relative to the first extension, so that the first extension, the second extension and part of the mounting body enclose a mounting slot;
the light-transmitting lens comprises a lens body configured to cover an orifice of the second mounting hole at one end facing the user's face, the lens body being provided with a flap at an edge, which is folded in a direction facing away from the user's face, the flap being inserted into the mounting slot.

10. The heart rate detection apparatus according to claim 1, wherein the first mounting hole is disposed in an upper portion of the face contact structure, and is located in a middle area of the face contact structure; and
the heart rate detection apparatus further comprises a protective cover, which detachably covers one end of the first mounting hole facing away from the user's face.

11. A head-mounted device, comprising:
a host; and
a heart rate detection apparatus, wherein the heart rate detection apparatus is connected to the host and comprises:
a face contact structure configured to abut against a user's face, the face contact structure comprising a first mounting hole located in an area of the face contact structure opposite to a user's forehead;
a mounting bracket disposed in the first mounting hole of the face contact structure; and
a heart rate sensor mounted on the mounting bracket and having a detection end disposed facing the user's face, the heart rate sensor being further electrically connected to a processor of the head-mounted device.

12. The head-mounted device according to claim 11, wherein the mounting bracket is slidably disposed in the first mounting hole;
the heart rate detection apparatus further comprises an elastic member having one end abutting against the mounting bracket, the elastic member being configured to push the mounting bracket and the heart rate sensor in a direction facing the user's face.

13. The head-mounted device according to claim 12, wherein in the first mounting hole is fixedly disposed a shaft seat and is further disposed a mounting shaft, one end of the mounting shaft being slidably disposed in a central hole of the shaft seat, and the other end of the mounting shaft being fixedly connected to the mounting bracket; one end of the elastic member facing away from the mounting bracket abuts against the shaft seat.

14. The head-mounted device according to claim 13, wherein the shaft seat comprises a mounting sleeve and a limit ring, wherein the limit ring is connected to one end of the mounting sleeve facing the elastic member; an aperture of a part of the central hole of the shaft seat located in the mounting sleeve is greater than that of a part of the central hole of the shaft seat located in the limit ring;
the mounting shaft is provided with a flange extending in its radial direction at one end facing the shaft seat, the flange being located in the mounting sleeve and having an outer diameter greater than the aperture of the part of the central hole of the shaft seat located in the limit ring.

15. The head-mounted device according to claim 11, wherein on one side of the face contact structure facing away from the user's face is further provided a quick release part, which is configured to detachably connect the face contact structure to a host of the head-mounted device;
wherein the quick release part comprises at least one of the following: a magnetic attraction member or a snap member.

16. The head-mounted device according to claim 15, further comprising a flexible circuit board, which is located at least in part in the first mounting hole and is electrically connected to the heart rate sensor, the flexible circuit board being further electrically connected to the processor of the head-mounted device through a pogo pin.

17. The head-mounted device according to claim 11, wherein the mounting bracket comprises a mounting body having a first end and a second end connected to each other, the first end being disposed facing the user's face; the first end being provided with a second mounting hole in which the heart rate sensor is located, the second end being provided with a shaft hole that is in sliding fit with one end of the mounting shaft facing the user's face.

18. The head-mounted device according to claim 17, wherein the mounting bracket is connected with a light-transmitting lens, which shields on one side of the heart rate sensor facing the user's face and at least covers an orifice of the second mounting hole.

19. The head-mounted device according to claim 18, wherein an outer periphery of the mounting body is further connected with a first extension extending outward, one side of which facing away from the mounting body is further connected with a second extension, the second extension being folded in the direction facing the user's face relative to the first extension, so that the first extension, the second extension and part of the mounting body enclose a mounting slot;
the light-transmitting lens comprises a lens body configured to cover an orifice of the second mounting hole at one end facing the user's face, the lens body being provided with a flap at an edge, which is folded in a direction facing away from the user's face, the flap being inserted into the mounting slot.

20. The head-mounted device according to claim 11, wherein the first mounting hole is disposed in an upper portion of the face contact structure, and is located in a middle area of the face contact structure; and
the heart rate detection apparatus further comprises a protective cover, which detachably covers one end of the first mounting hole facing away from the user's face.
